(19) United States
(12) Patent Application Publication
(10) Pub. No.: US 2007/0081954 A1
(43) Pub. Date: Apr. 12, 2007

(54) Dispersion of polymer particles, composition comprising it and cosmetic treatment method

(76) Inventors: Nathalie Mougin, Paris (FR); Gwenaelle Jegou, Livry Gargan (FR); Franck Giroud, Clichy (FR); Henri Samain, Bievres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/543,115

(22) Filed: Oct. 5, 2006

Related U.S. Application Data
(60) Provisional application No. 60/727,515, filed on Oct. 18, 2005.

(30) Foreign Application Priority Data
Oct. 6, 2005 (FR) 05 53032

(51) Int. Cl. A61K 8/89; A61K 8/81

(52) U.S. Cl. 424/61; 424/70.12; 424/70.1

(57) ABSTRACT

The present patent application relates to novel dispersions of particles of a polymer which is stabilized at the surface, in a nonaqueous medium, in which dispersions the polymer exhibits a specific glass transition temperature. Another aspect of the present disclosure is a cosmetic or pharmaceutical composition comprising the said dispersion in a cosmetically or pharmaceutically acceptable medium. The present disclosure also relates to a method for the cosmetic treatment of keratinous substances employing the presently disclosed composition.

This application claims benefit of U.S. Provisional Application No. 60/727,515, filed Oct. 18, 2005, the contents of which are incorporated herein by reference. This application also claims benefit of priority under 35 U.S.C. § 119 to French Patent Application No. FR 05 53032, filed Oct. 6, 2005, the contents of which are also incorporated herein by reference.

The present disclosure relates to a novel dispersion of particles of highly specific polymers which are dispersed in a nonaqueous medium and to the compositions, such as cosmetic or pharmaceutical compositions, comprising the dispersion.

Non-limiting mention may be made, among the polymers employed in the cosmetics field and including those employed in the hair field, of cationic polymers, such as those based on dimethyldiallylammonium chloride, known for protecting and/or beautifying the hair for instance, by virtue of their high substantivity. However, no hair-shaping effect is observed when polymers of this type are employed. Furthermore, their incompatibility with the majority of propellants does not allow them to be used in aerosol products, such as lacquers.

The document EP 1 323 753 discloses hair compositions comprising aqueous dispersions of hydrophobic cationic polymers which contribute styling properties, for example when they are employed in shampoos. However, these compositions do not always exhibit very good cosmetic properties in a wet medium, for example as the shampoos are being applied.

In order to obtain a good styling effect while retaining acceptable cosmetic properties in the compositions, provision has been made for polymers having styling properties conveyed in a cosmetic solvent. Thus, in the documents WO 91/15185 and WO 98/18433, provision is made for cosmetic compositions comprising hydrophobic or water-insoluble polymers conveyed in solution in an organic solvent. However, the need to employ polymers soluble in an organic medium implies a low variability in the chemical structures available.

Furthermore, it has been found that organic solutions of hydrophobic polymers generally exhibit a high viscosity, related to the amount of polymer in the solution, which makes the subsequent formulation of these polymers and of their solutions difficult.

Furthermore, the use of dispersions of polymer particles in cosmetics is understood by those in the art, the particles generally being of nanometric size, in organic media and for instance, as taught by EP 749 747, of nonaqueous dispersions of poly(methyl acrylate) or poly(methyl methacrylate) particles in a non-volatile liquid paraffin or in isododecane, for example. However, these dispersions do not make it possible to obtain satisfactory cosmetic properties, in terms of styling effect and of disentangling, for example. Furthermore, the feel of the compositions comprising them can prove to be not very satisfactory, with regard to softness, for instance.

The present inventors have discovered, surprisingly, novel dispersions of polymer particles which are stabilized by stabilizing agents in nonaqueous media which make it possible to contribute at least one of the desired styling properties while improving at least one of the cosmetic properties (feel, softness, disentangling).

A subject-matter of the present disclosure is thus a dispersion of particles of at least one ethylenic polymer which is stabilized at its surface by a stabilizing agent in a nonaqueous medium comprising at least one nonaqueous compound, liquid at 25° C., having an overall solubility parameter according to the Hansen solubility space of less than or equal to 20 (MPa)^{1/2}, or a mixture of such compounds; wherein the at least one ethylenic polymer exhibits a glass transition temperature (Tg) of less than or equal to −20° C.

Another aspect of the present disclosure is a cosmetic or pharmaceutical composition comprising, in a cosmetically or pharmaceutically acceptable medium, at least one dispersion as defined above.

The present disclosure makes it possible to prepare polymers which can be easily conveyed, given that the dispersions have relatively low viscosities, which facilitate the use thereof in cosmetic compositions.

Furthermore, these compositions make it possible to confer body and hold on the hair.

In addition, these dispersions or compositions comprising them contribute beneficial properties, such as in the rinse-out mode. They make it possible to obtain, in addition to the styling effects and the hold, a soft and nontacky feel, good softness, and also ease of disentangling of the hair, in a dry and/or wet environment.

These polymers can also be used in “leave-in” products of styling gel/foam or lacquer type and can contribute, in addition to styling, cosmetic properties to the hair (feel, softness, smoothing, disentangling).

Another benefit of the present disclosure lies in the fact that the polymer particles can be of very small size, for instance nanometric size, which is not the case with, for example, other types of particles, such as microspheres, the diameter of which is generally greater than 1 micron. In point of fact, a large size, of the order of a micron, has the disadvantage of bringing about a degree of visibility of the particles to the eye, when they are in a composition and when they are applied to the skin, and results in poor stability of the composition over time, for example. Thus, the dispersions according to the present disclosure make it possible to obtain stable compositions which can in addition be transparent, translucent or opaque, for instance, according to the size of the polymer particles which are dispersed therein.

The dispersions according to the present disclosure are thus composed of particles, generally spherical particles, of at least one ethylenic polymer which is stabilized at the surface by a stabilizing agent in a nonaqueous medium.

For instance, the dispersions according to the present disclosure can be provided in the form of polymer nanoparticles in stable dispersion in a nonaqueous medium. The nanoparticles may have a size ranging from 5 to 600 nm, such as 10 to 500 nm, for instance 15 to 450 nm, given that, beyond approximately 600 nm, the dispersions of particles become much less stable.

For example, these particles remain in the form of individual particles, without forming agglomerates, when they are in dispersion in the nonaqueous media.

As disclosed herein, the term “ethylenic” polymer is generally understood to mean a polymer obtained by polymerization of at least 2 identical or different monomers comprising an ethylenic unsaturation.

The at least one ethylenic polymer can be chosen by a person skilled in the art as a function of its properties, according to the desired subsequent application of the composition. These polymers may be crosslinked, for example.

However, the at least one ethylenic polymer may exhibit at least one glass transition temperature (Tg) of less than or equal to −20° C., for example, ranging from −150° C. to −20° C., such as ranging from −99° C. to −25° C., for instance from −95° C. to −30° C., even from −80° C. to −35° C. and further still, for example, from −70° C. to −40° C.

As mentioned, for instance, the polymer may exhibit only one glass transition temperature. However, it may also exhibit several glass transition temperatures, for example two Tg values; in this case, the lowest Tg must be less than −20° C.

In the present disclosure, the Tg values (or glass transition temperatures) indicated are theoretical Tg values determined from the theoretical Tg values of the constituent monomers of the polymer, which can be found in a reference handbook, such as the Polymer Handbook, 3^{rd} ed., 1989, John Wiley, according to the following relationship, referred to as the Fox law:

$$\frac{1}{Tg} = {\sum }_{i}^{} ( \frac{ϖi}{Tgi} )$$

wherein:
ω̅i is the mass fraction of the monomer i in the polymer and Tgi is the glass transition temperature of the homopolymer of the monomer i, expressed in degrees Kelvin (K).

According to the present disclosure, the term “monomer with a Tg” is generally understood to mean the monomer, the homopolymer of which has such a glass transition temperature.

The polymers according to the present disclosure can be linear, branched or even star homopolymers or copolymers. They can be random or alternating. In at least one embodiment, they are random linear copolymers.

Generally, the monomers with a Tg of less than or equal to −20° C. can be present in an amount ranging from 50 to 100% by weight relative to the total weight of the starting monomers.

According to at least one embodiment of the present disclosure, the polymers present in the dispersion can result from the polymerization of at least one monomer with a Tg of less than or equal to −20° C., for instance ranging from −150° C. to −20° C., for example ranging from −99° C. to −25° C., such as, for instance, ranging from −95° C. to −30° C., indeed even ranging from −80° C. to −40° C. and also −70° C. to −45° C.

In this embodiment, the monomer with a Tg of less than or equal to −20° C., or the mixture thereof, is present in an amount of 100% by weight relative to the total weight of the starting monomers.

According to another embodiment of the present disclosure, the polymers present in the dispersion can result from the polymerization of at least one monomer with a Tg of less than or equal to −20° C. and at least one “additional” monomer with a Tg of greater than −20° C. but present in an amount such that the overall Tg of the polymer is less than or equal to −20° C.

For example, it is possible to combine, in the final polymer, a monomer with a Tg of the order of 100° C., which can be present in a proportion of 10-20% by weight relative to the total weight of monomers, and a monomer with a Tg of the order of −50° C., which can be present in a proportion of 80-90% by weight, so as to obtain a polymer having a Tg from −40° C. to −30° C.

In this embodiment, the at least one additional monomer, or the mixture thereof can be present in an amount from 0.01 to 50% by weight, with respect to the total weight of the monomers, for instance from 0.1 to 40% by weight, indeed even from 1 to 30% by weight or also from 5 to 15% by weight relative to the total weight of the monomers. The monomer with a Tg of less than or equal to −20° C., or the mixture of such monomers, can then be present in a proportion from 50 to 99.99% by weight, for example from 60 to 99.9% by weight, indeed even from 70 to 99% by weight or also from 85 to 95% by weight, with respect to the total weight of monomers.

A person skilled in the art will know, on the basis of the Fox law and general knowledge known to those in the art, how to determine the maximum amounts of additional monomer that may be present in the polymer of the dispersion, so as to always obtain, at the end, a dispersion of polymer having a Tg of less than or equal to −20° C.

Furthermore, it has been found that, when the polymer dispersion according to the present disclosure comprises at least one “hydrophobic” monomer as defined below, it exhibits good properties of persistence of the deposited layer, and of softness, and good compatibility with compositions of conditioner type.

Thus, for example, the at least one ethylenic polymer according to the present disclosure may comprise from 40% to 100% by weight, with respect to the total weight of monomers, for example from 60 to 99% by weight, indeed even from 70 to 98% by weight and further still, for example, from 60 to 95% by weight, of hydrophobic monomer, alone or as a mixture.

The at least one hydrophobic monomer can be chosen from monomers having a Tg of less than or equal to −20° C. and/or from monomers having a Tg of greater than −20° C.

For instance, the at least one hydrophobic monomer may have a Tg of less than −20° C.

The term “hydrophobic monomer” is generally understood, within the meaning of the present disclosure, to mean a monomer having a value of the logarithm of the 1 -octanol/water apparent partition coefficient, also referred to as log P, of greater than or equal to 2, for example ranging from 2 to 11, for instance ranging from 2.5 to 10, for example ranging from 3 to 8, indeed even ranging from 3.5 to 5.

The log P values are known and are determined according to a standard test which determines the concentration of the monomer in 1-octanol and water.

For instance, the values can be calculated using the ACD (Advanced Chemistry Development) software Solaris V4.67; they can also be obtained from Exploring QSAR: hydrophobic, electronic and steric constants (ACS professional reference book, 1995). There is also an Internet site which provides estimated values (address: http://esc.syrres.com/interkow/kowdemo.htm).

The value of log P for some standard monomers, determined using the ACD software, is shown below:

| | Acrylate | Methacrylate |
|---|---|---|
| | (*acrylamide) | (*methacrylamide) |
| Methyl (meth)acrylate | 0.793 +/− 0.223 | 1.346 +/− 0.250 |
| Ethyl (meth)acrylate | 1.325 +/− 0.223 | 1.877 +/− 0.250 |
| Propyl (meth)acrylate | 1.856 +/− 0.223 | 2.408 +/− 0.250 |
| Isopropyl (meth)acrylate | 1.672 +/− 0.228 | 2.224 +/− 0.254 |
| n-Butyl (meth)acrylate | 2.387 +/− 0.223 | 2.940 +/− 0.250 |
| Isobutyl (meth)acrylate | 2.208 +/− 0.228 | 2.756 +/− 0.254 |
| tert-Butyl (meth)acrylate | 2.022 +/− 0.238 | 2.574 +/− 0.261 |
| Cyclohexyl(meth)acrylate | 2.853 +/− 0.226 | 3.405 +/− 0.252 |
| Octyl (meth)acrylate | 4.513 ± 0.224 | 5.065 +/− 0.521 |
| Lauryl (meth)acrylate | 6.638 ± 0.224 | 7.190 +/− 0.251 |
| Tridecyl (meth)acrylate | 7.170 ± 0.224 | 7.712 ± 0.251 |
| Cetyl(meth)acrylate | 8.764 ± 0.224 | 9.316 ± 0.251 |
| Palmityl (meth)acrylate | >9 | >9 |
| Stearyl (meth)acrylate | 9.826 ± 0.224 | 10.379 ± 0.251 |
| Behenyl (meth)acrylate | 12.504 ± 0.251 | 11.952 ± 0.225 |
| Oleyl (meth)acrylate | 9.308 ± 0.232 | >9 |
| Tetrahydrofurfuryl | 0.800 ± 0.263 | 1.352 ± 0.283 |
| (meth)acrylate | | |
| 2-Ethylhexyl (meth)acrylate | 4.329 ± 0.229 | 4.881 ± 0.254 |
| 2-Hydroxyethyl (meth)acrylate | 0.166 ± 0.258 | 0.718 ± 0.277 |
| Ethoxyethyl (meth)acrylate | 1.335 ± 0.268 | 1.887 ± 0.293 |
| Hydroxypropyl (meth)acrylate | 0.383 ± 0.241 | |
| Dimethylaminoethyl | | 0.97 |
| (meth)acrylate (MADAME) | | |
| N-Isopropyl(meth)acrylamide* | 0.195 ± 0.256 | 0.748 ± 0.276 |
| N-Octyl(meth)acrylamide* | 3.036 ± 0.253 | 3.558 ± 0.273 |
| N-(tert-Butyl)(meth)acrylamide* | 1.02 | |
| N,N- | −0.168 ± 0.556 | 0.906 ± 0.553 |
| Dimethyl(meth)acrylamide* | | |
| N,N-Dibutyl(meth)acrylamide* | 3.021 ± 0.557 | 3.573 ± 0.570 |
| (Meth)acrylic acid | 0.35 | 0.83 |

Among the monomers with a Tg of less than or equal to −20° C. capable of being employed in forming the dispersion according to the present disclosure, non-limiting mention may be made of:
(i) esters of acrylic acid of formula CH₂═CHCOOR1 where R1 is (a) a saturated or unsaturated, and linear or branched carbon chain, such as a C₂-C₁₂ hydrocarbon (alkyl) chain, optionally comprising, inserted, at least one heteroatom chosen from O, N, S; and/or optionally substituted by at least one substituent chosen from —OH and halogen atoms (Cl, Br, I and F), with the exception of the tert-butyl chain; or else R1 is (b) a polyoxyethylene group comprising from 5 to 30 ethylene oxide units; or also R1 is (c) a —R—(OC₂H₄)ₙ—H group with R═C₁-C₁₂ alkyl and n is an integer from 5 to 30 inclusive.

Non-limiting mention may thus be made of ethyl, propyl, n-butyl, isobutyl, 2-ethylhexyl, octyl, isooctyl, isodecyl, decyl, lauryl, tridecyl, hydroxyethyl and hydroxypropyl acrylates.
(ii) esters of methacrylic acid of formula CH₂═C(CH₃)COOR2 where R2 is (a) a saturated or unsaturated, and linear or branched carbon chain, such as a C₈-C₁₂ hydrocarbon (alkyl) chain, optionally comprising, inserted, at least one heteroatom chosen from O, N, S; and/or optionally substituted by at least one substituent chosen from —OH and halogen atoms (Cl, Br, I and F); or else R2 is (b) a polyoxyethylene group comprising from 5 to 30 ethylene oxide units; or also R2 is (c) a —R—(OC₂H₄)ₙ—H group with R═C₁-C₃₀ alkyl and n is an integer from 5 to 30 inclusive.

Non-limiting mention may thus be made of octyl, isooctyl, decyl, isodecyl, dodecyl, lauryl, tridecyl, myristyl, cetyl, palmityl, stearyl, behenyl and oleyl methacrylates.
(iii) vinyl esters of formula CH₂═CH—OCO—R3 where R3 is a saturated or unsaturated, and linear or branched carbon chain, for instance a C₂-C₁₂ hydrocarbon chain, among which non-limiting mention may be made of vinyl butyrate (or butanoate), vinyl ethylhexanoate, vinyl neononanoate and vinyl neododecanoate;
(iv) vinyl ethers of formula CH₂═CHOR4 where R4 is a saturated or unsaturated and linear or branched carbon chain, for instance a C₁-C₁₂ hydrocarbon chain, among which non-limiting mention may be made of vinyl ether, methyl vinyl ether, ethyl vinyl ether, ethylhexyl vinyl ether and butyl vinyl ether;
(v) N-alkyl(meth)acrylamides of formula CH₂═CHCONR5R′5 or CH₂═C(CH₃)CONR5R′5 where R5 and R′5 are, independently of one another, a hydrogen atom or a saturated or unsaturated and linear, cyclic or branched carbon chain, such as a C₆-C₂₈ hydrocarbon chain, optionally aromatic chain (aryl, aralkyl or alkylaryl), optionally comprising, inserted, at least one heteroatom chosen from O, N, S; and/or optionally substituted by at least one substituent chosen from —OH and halogen atoms (Cl, Br, I and F); given that at least one of the R5 and R′5 radicals is other than hydrogen;
among which non-limiting mention may be made of N-octylacrylamide and N-octadecylacrylamide.

The monomers with a Tg of less or equal to −20° C. include but are not limited to n-butyl acrylate, ethylhexyl acrylate, isobutyl acrylate, isooctyl acrylate and mixtures thereof.

Non-limiting mention may be made, among the monomers having a Tg of less than or equal to −20° C. and furthermore being hydrophobic, of n-butyl, isobutyl, 2-ethylhexyl, octyl, isooctyl, isodecyl, decyl and lauryl acrylates; and also octyl, isooctyl, decyl, isodecyl, dodecyl, lauryl, tridecyl, cetyl, palmityl, stearyl and oleyl methacrylates.

As is mentioned above, the polymer in dispersion according to the present disclosure can comprise at least one additional monomer with a Tg of greater than −20° C., for instance greater than 0° C., with the proviso that the at least one additional monomer and their amount are chosen so that the overall Tg of the polymer is less than or equal to −20° C.

The at least one additional monomer can be chosen from the following monomers:
(i) vinyl compounds of formula CH₂═CHR6 in which R6 is
a hydroxyl group;
a C₁ to C₂₅ linear or branched alkyl group in which is optionally inserted at least one heteroatom chosen from O, N, S and P; it being possible for said alkyl group in addition to be optionally substituted by at least one substituent chosen from —OH and halogen atoms (Cl, Br, I and F);
a C₃ to C₈ cycloalkyl group, such as cyclohexyl,
a C₆ to C₂₀ aryl group, such as phenyl,
a C₇ to C₃₀ aralkyl group (C₁ to C₄ alkyl group), such as 2-phenylethyl or benzyl,
a 4- to 12-membered heterocyclic group comprising at least one heteroatom chosen from O, N and S,
a heterocycloalkyl group (alkyl of 1 to 4 carbons), such as furfuryl, furfurylmethyl or tetrahydrofurfurylmethyl,
   it being possible for said cycloalkyl, aryl, aralkyl, heterocyclic or heterocycloalkyl groups to be optionally substituted by at least one substituent chosen from the hydroxyl group, halogen atoms and linear or branched C₁-C₄ alkyl groups in which is optionally inserted at least one heteroatom chosen from O, N, S and P, it being possible for said alkyl groups in addition to be optionally substituted by at least one substituent chosen from —OH and halogen atoms (Cl, Br, I and F).

Non-limiting examples of vinyl monomers include but are not limited to vinylcyclohexane, styrene and vinyl acetate.
(ii) acrylates of formula CH₂═CHCOOR7 in which R7 is a tert-butyl group; a C₃ to C₈ cycloalkyl group; a C₆ to C₂₀ aryl group; a C₇ to C₃₀ aralkyl group (C₁ to C₄ alkyl group); a 4- to 12-membered heterocyclic group comprising at least heteroatom chosen from O, N and S; a heterocycloalkyl group (C₁ to C₄ alkyl), such as a furfuryl group; it being possible for the said cycloalkyl, aryl, aralkyl, heterocyclic or heterocycloalkyl groups to be optionally substituted by at least one substituent chosen from the hydroxyl group, halogen atoms and linear or branched C₁-C₄ alkyl groups in which is optionally inserted at least one heteroatom chosen from O, N, S and P, it being possible for the said alkyl groups in addition to be optionally substituted by at least one substituent chosen from the hydroxyl group and halogen atoms (Cl, Br, I and F).

Non-limiting examples of such monomers include but are not limited to tert-butyl, t-butylcyclohexyl, t-butylbenzyl, furfuryl and isobornyl acrylates, for instance.
(iii) methacrylates of formula CH₂═C(CH₃)COOR8 in which R8 is:
a linear or branched carbon group, for instance a C₁-C₆ hydrocarbon (alkyl) group, such as a methyl, ethyl, propyl or isobutyl group, it being possible for the said alkyl group in addition to be optionally substituted by at least one substituent chosen from OH and halogen atoms (Cl, Br, I and F);
a C₃ to C₈ cycloalkyl group;
a C6 to C20 aryl group;
a C₇ to C₃₀ aralkyl group (C₁ to C₄ alkyl group);
a 4- to 12-membered heterocyclic group comprising at least one heteroatom chosen from O, N and S;
a heterocycloalkyl group (C₁-C₄ alkyl), such as a furfuryl group;
   it being possible for the said cycloalkyl, aryl, aralkyl, heterocyclic or heterocycloalkyl groups to be optionally substituted by at least one substituent chosen from OH, halogen atoms and linear or branched C₁-C₄ alkyl groups in which is optionally inserted at least one heteroatom chosen from O, N, S and P, it being possible for the said alkyl groups in addition to be optionally substituted by at least one substituent chosen from hydroxyl groups and halogen atoms (Cl, Br, I and F).

Non-limiting examples of such monomers include but are not limited to methyl, ethyl, propyl, n-butyl, isobutyl, t-butylcyclohexyl, t-butylbenzyl, methoxyethyl, methoxypropyl and isobornyl methacrylates.
(iv) (meth)acrylamides of formula CH₂═CHCONR9R′9 or CH₂═C(CH₃)CONR9R′9 in which R9 and R′9, which are identical or different, represent a hydrogen atom or a linear or branched C₁-C₅ alkyl group, such as an n-butyl, t-butyl or isopropyl group.

Non-limiting examples of such monomers include but are not limited to N-butyl(meth)acrylamide, N-isopropyl(meth)acrylamide, N,N-dimethyl(meth)acrylamide and N,N-dibutyl(meth)acrylamide.

For instance, the monomers with a Tg of greater than −20° C. include but are not limited to furfuryl, isobornyl, tert-butyl, tert-butylcyclohexyl and tert-butylbenzyl acrylates; methyl, n-butyl, ethyl and isobutyl methacrylates, styrene, vinyl acetate and vinylcyclohexane, and their mixtures.

Among the monomers having a Tg of greater than −20° C. and furthermore being hydrophobic, non-limiting mention may be made of isobornyl, tert-butyl, tert-butylcyclohexyl and tert-butylbenzyl acrylates; n-butyl and isobutyl methacrylates, styrene.

The polymer present in the dispersion according to the present disclosure can, of course, comprise hydrophilic monomers (that is to say, nonhydrophobic monomers or also monomers having a log P of less than 2) which can have a Tg of less than or equal to −20° C. or a Tg of greater than −20° C., or a mixture of such hydrophilic monomers.

Non-limiting mention may be made, among the hydrophilic monomers capable of being employed, of acrylic acid, methacrylic acid, dimethylaminoethyl methacrylate (MADAME), dimethylaminopropylmethacrylamide (DMAPMA), vinylpyridine and vinylimidazole, MADQUAT (or [2-(methacryloyloxy)ethyl]trimethylammonium chloride), MAPTAC (or methacrylamidopropyltrimethylammonium chloride), 2-hydroxyethyl methacrylate, 2-hydroxyethyl acrylate, N-(tert-butyl)acrylamide, tetrahydrofurfuryl methacrylate, methyl methacrylate, ethyl methacrylate, methyl acrylate, ethyl acrylate, vinylpyrrolidone, vinyl acetate, acrylates or methacrylates of formula CH₂═C(H,CH₃)COOR with R alkyl which can comprise (OC₂H₄)ₘ—OR″ groups, with m=5 to 150 and R″═H or C₁ to C₃₀ alkyl.

When it comprises acid functional groups, the polymer according to the present disclosure can be neutralized by an organic base, for example a primary, secondary or tertiary amine, it being possible for the amine to comprise or not comprise substituents (hydroxyl), such as 2-amino-2-methylpropanol, and the salified or quaternized forms of these.

When it comprises basic functional groups, the polymer according to the present disclosure can be neutralized by organic acids which can comprise at least one carboxyl, sulpho or phosphono groups. They can be linear, branched or cyclic aliphatic acids or also aromatic acids. These acids can additionally comprise at least one heteroatom chosen from O and N, for example in the form of hydroxyl groups. Non-limiting mention may be made of acetic acid, α-hydroxyethanoic acid, α-hydroxyoctanoic acid, α-hydroxycaprylic acid, ascorbic acid, benzoic acid, behenic acid, capric acid, caproic acid, caprylic acid, citric acid, dodecylbenzenesulphonic acid, 2-ethylcaproic acid, folic acid, fumaric acid, galactaric acid, gluconic acid, glycolic acid, 2-hexadecyleicosanoic acid, hydroxycaproic acid, 12-hydroxystearic acid, isolauric (or 2-butyloctanoic) acid, isomyristic (or 2-hexyloctanoic) acid, isoarachidic (or 2-octyldodecanoic) acid, isolignoceric (or 2-decyltetradecanoic) acid, lactic acid, lauric acid, malic acid, myristic acid, oleic acid, palmitic acid, propionic acid, sebacic acid, stearic acid, tartaric acid, terephthalic acid, trimesic acid, undecylenic acid, propyl betaine, cocoamidopropyl betaine, and their mixtures.

For example, the polymers which can be used within the context of the present disclosure may have a number-average molecular weight (Mn) ranging from 2,000 to 1,000,000, for example ranging from 3,000 to 800,000, and further still for example ranging from 4,000 to 500,000.

The dispersion of polymer particles according to the present disclosure thus comprises a nonaqueous medium in which the particles are dispersed.

This nonaqueous medium is composed of at least one nonaqueous compound, liquid at 25° C., having an overall solubility parameter according to the Hansen solubility space of less than or equal to 20 (MPa)^{1/2}, or a mixture of such compounds.

The overall solubility parameter δ according to the Hansen solubility space is defined in the article “Solubility parameter values” by Grulke in the work “Polymer Handbook”, 3^{rd} edition, Chapter VII, pages 519-559, by the relationship:
δ=(d_{D}²+d_{P}²+d_{H}²)^{1/2}
in which:
d_{D} denotes the London dispersion forces resulting from the formation of dipoles induced during molecular impacts,
d_{P} denotes the forces of Debye interactions between permanent dipoles,
d_{H} denotes the forces of specific interactions (hydrogen bond, acid/base or donor/acceptor type, and the like).

The definition of the solvents in the three-dimensional solubility space according to Hansen is described in the paper by Hansen: “The three-dimensional solubility parameters”, J. Paint Technol., 39, 105 (1967).

Non-limiting mention may be made, among the nonaqueous liquid compounds having an overall solubility parameter according to the Hansen solubility space of less than or equal to 20 (MPa)^{1/2}, of liquid fatty substances, such as oils, which can be chosen from optionally branched, natural or synthetic, carbon, hydrocarbon, fluorinated and silicone oils, alone or as a mixture.

Non-limiting mention may be made, among these oils, of vegetable oils formed by esters of fatty acids and of polyols, for instance triglycerides, such as sunflower, sesame or rapeseed oil, or esters derived from long-chain (as disclosed herein, generally understood as having from 6 to 20 carbon atoms) acids or alcohols, such as esters of formula RCOOR′ in which R is the residue of a higher fatty acid comprising from 7 to 19 carbon atoms, and R′ is a hydrocarbon chain comprising from 3 to 20 carbon atoms, such as palmitates, adipates, myristates and benzoates, for example diisopropyl adipate and isopropyl myristate.

Non-limiting mention may also be made of hydrocarbons, for example liquid paraffin, liquid petrolatum, or hydrogenated polyisobutylene, C₈-C₁₆ isoparaffins and volatile isoparaffins, such as isododecane or the “Isopars”.

Non-limiting mention may also be made of silicone oils, such as polydimethylsiloxanes and polymethylphenylsiloxanes, which are optionally substituted by aliphatic and/or aromatic groups which are optionally fluorinated or by functional groups, such as hydroxyl, thiol and/or amine groups; and volatile silicone oils, for example cyclic or linear volatile silicone oils, such as cyclodimethylsiloxanes, cyclophenylmethylsiloxanes and linear dimethylsiloxanes, among which may be mentioned linear dodecamethylpentasiloxane (L5), octamethylcyclotetrasiloxane, decamethylcyclopentasiloxane, hexadecamethylcyclohexasiloxane, heptamethylhexyltrisiloxane or heptamethyloctyltrisiloxane.

Non-limiting mention may also be made of solvents, alone or as a mixture, chosen from linear, branched or cyclic esters having 6 to 30 carbon atoms; ethers having 6 to 30 carbon atoms and ketones having 6 to 30 carbon atoms.

Non-limiting mention may also be made, among the nonaqueous compounds capable of being employed, of monoalcohols having an overall solubility parameter according to the Hansen solubility space of less than or equal to 20 (MPa)^{1/2}, that is to say aliphatic fatty monoalcohols having at least 6 carbon atoms, for instance 6 to 32, the hydrocarbon chain not comprising a substituent group. Further non-limiting mention may be made, as monoalcohols according to the present disclosure, of oleyl alcohol, decanol, dodecanol, octadecanol and linoleyl alcohol.

For instance, according to at least one embodiment, the nonaqueous medium comprises volatile silicone oils, such as cyclic or linear volatile silicone oils, such as cyclodimethylsiloxanes and linear dimethylsiloxanes, and/or esters of formula RCOOR′ in which R is the residue of a higher fatty acid comprising from 7 to 19 carbon atoms and R′ is a hydrocarbon chain comprising from 3 to 20 carbon atoms, such as palmitates, adipates, myristates and benzoates, for example diisopropyl adipate and isopropyl myristate; and their mixtures.

The choice of the nonaqueous medium can be easily made by a person skilled in the art according to the nature of the monomers constituting the polymer and/or the nature of the stabilizing agent.

The polymer dispersion can be manufactured as disclosed in the document EP-A-749 747. Generally, the polymerization can be carried out in dispersion, that is to say by precipitation of the polymer during formation, with protection using a stabilizing agent of the particles formed.

It is thus possible to prepare a mixture comprising the starting monomers and a radical initiator. This mixture is dissolved in a synthesis solvent.

The monomers are soluble in the reaction medium, whereas the polymer is not soluble therein. As the polymerization takes place, the polymer will precipitate and will be found stabilized by the stabilizing agent present. Polymer particles protected at the surface by the stabilizing agent are thus obtained.

The polymerization can be carried out directly in the nonaqueous medium, which can thus also act as synthesis solvent.

It is also possible to carry out the polymerization in a synthesis solvent and then subsequently to carry out a solvent exchange, the synthesis solvent being replaced by the nonaqueous medium.

Thus, when the nonaqueous medium chosen is a nonvolatile hydrocarbon or silicone oil, the polymerization can be carried out in a nonpolar organic solvent (synthesis solvent), then the nonvolatile oil (which must be miscible with the said synthesis solvent) is added and the synthesis solvent is selectively distilled.

According to at least one embodiment, a synthesis solvent is chosen such that the starting monomers and the radical initiator are soluble therein and the particles of polymer obtained are insoluble therein in order for them to precipitate therein during their formation. For example, the synthesis solvent can be chosen from alkanes, such as heptane, isododecane or cyclohexane.

When the nonaqueous medium chosen is a volatile hydrocarbon or silicone oil, the polymerization can be carried out directly in the said oil, which thus also acts as synthesis solvent. For instance, the monomers should be soluble therein, as well as the radical initiator, and the polymer obtained must be insoluble therein.

According to at least one embodiment, the monomers are present in the synthesis solvent, before polymerization, in an amount ranging from 5 to 80% by weight relative to the total weight of the starting mixture of monomers. All of the monomers may be present in the solvent before the beginning of the reaction or a portion of the monomers may be added as the polymerization reaction progresses.

The radical initiator can, for example, be an azo or peroxide compound, such as azobisisobutyronitrile or tert-butyl peroxy(2-ethylhexanoate).

The polymer particles can be stabilized at the surface.

In a first embodiment, the particles can be stabilized at the surface as the polymerization occurs, by virtue of a stabilizing agent which can, in at least one embodiment, be a sequential polymer, a grafted polymer and/or a random polymer, alone or as a blend. Stabilization can be carried out by any known means, for instance by polymerization in the presence of the stabilizing agent.

For example, the stabilizing agent can be present in the mixture at the start of the polymerization. However, it is also possible to add it continuously, such as when monomers are also being added continuously.

In a second embodiment, the polymer can be synthesized in a synthesis solvent and then dispersed in a nonaqueous dispersing medium by addition of the dispersing agent, and the synthesis solvent is evaporated.

Use may be made of a stabilizing agent in an amount ranging from 0.1 to 30% by weight, with respect to the weight of the starting mixture of monomers, for instance from 1 to 20% by weight, such as from 2 to 15% by weight, indeed even from 3 to 10% by weight, for example.

When a grafted and/or sequential polymer is used as stabilizing agent, the synthesis solvent is ideally chosen so that at least a portion of the grafts or sequences of the stabilizing polymer is soluble in the solvent, the other portion of the grafts or sequences not being soluble therein. The stabilizing polymer used during the polymerization must be soluble or dispersible in the synthesis solvent. Furthermore, according to at least one embodiment, the stabilizing agent used comprises a portion (sequences, grafts or other) exhibiting a degree of affinity for the polymer formed during the polymerization.

When a random polymer is used as stabilizing agent, it should be chosen so that the stabilizing agent has a sufficient amount of groups rendering it soluble in the synthesis solvent envisaged.

Among the grafted polymers, non-limiting mention may be made of silicone polymers grafted with a hydrocarbon chain and hydrocarbon polymers grafted with a silicone chain.

Grafted copolymers having, for example, an insoluble backbone of polyacrylic type with soluble grafts of poly(12-hydroxystearic acid) type are also suitable.

Non-limiting mention may also be made, as the stabilizing polymer, of:
(a) grafted or sequential block copolymers comprising at least one block of polyorganosiloxane type and at least one block of a polymer (i) resulting from radical polymerization or (ii) resulting from polycondensation, such as polyether, polyester or polyamide type, or their blend, it being possible for the copolymer to comprise fluorinated entities.

Non-limiting mention may be made, as grafted or sequential block copolymers comprising at least one block of polyorganosiloxane type and at least one block of a radical polymer, of grafted copolymers of acrylic/silicone type which can be employed for instance when the nonaqueous medium is a silicone medium.

When the grafted or sequential block copolymers comprise at least one block of polyorganosiloxane type and at least one polyether block, the polyorganosiloxane block can be, for example, a polydimethylsiloxane or alternatively a poly(C₂-C₁₈)alkyl-methylsiloxane; the polyether block can be a polyoxy(C₂-C₁₈)alkylene, such as polyoxyethylene and/or polyoxypropylene. Use may thus be made of dimethicone copolyols or also (C₂-C₁₈)alkyl methicone copolyols which are optionally crosslinked. Use may be made, for example, of the dimethicone copolyol sold under the name “Dow Corning 3225C” by Dow Corning or the lauryl methicone copolyol sold under the name “Dow Corning Q2-5200” by Dow Corning.

Non-limiting mention may also be made of lauryl dimethicone copolyol crosspolymer, for example KSG31 or KSG32 from Shin-Etsu, cetyl dimethicone copolyol, such as DMC 3071 from GE, and dimethicone copolyol PPG-3 oleyl ether, such as KF-6026 from Shin-Etsu.
(b) grafted or sequential block copolymers of C₁-C₄ alkyl (meth)acrylates and of C₈-C₃₀ alkyl(meth)acrylates. Non-limiting mention may be made of the stearyl methacrylate/methyl methacrylate copolymer.
(c) grafted or sequential block copolymers comprising at least one block resulting from the polymerization of ethylenic monomer, comprising at least one optionally conjugated ethylenic bonds, such as of diene; and at least one block of polymer resulting from radical polymerization other than diene, for example resulting from vinyl, (meth)acrylic or (meth)acrylamide monomer, or of a polyether, of a polyester or of a polyamide, or their blends.

For instance, use may be made of copolymers comprising at least one block resulting from the polymerization of at least one ethylenic monomer, comprising at least one optionally conjugated ethylenic bonds, such as ethylene, butadiene or isoprene, and at least one block of a styrene polymer. When the ethylenic monomer comprises several optionally conjugated ethylenic bonds, the residual ethylenic unsaturations after the polymerization are generally hydrogenated. Thus, in a known way, the polymerization of isoprene results, after hydrogenation, in the formation of an ethylene-propylene block and the polymerization of butadiene results, after hydrogenation, in the formation of an ethylene-butylene block.

Non-limiting mention may be made, among these sequential copolymers, of copolymers of “diblock” or “triblock” type of the polystyrene/polyisoprene or polystyrene/polybutadiene type, such as those sold under the name of “Luvitol HSB” by BASF, of the polystyrene/copoly(ethylene-propylene) type, such as those sold under the name of “Kraton” by Shell Chemical Co., or also of the polystyrene/copoly(ethylene-butylene) type.

Non-limiting mention may be made, as grafted or sequential block copolymers comprising at least one block resulting from the polymerization of at least one ethylenic monomer, such as ethylene or isobutylene, and at least one block of an acrylic polymer, such as methyl methacrylate, of poly(methyl methacrylate)/polyisobutylene bi- or trisequential copolymers or grafted copolymers comprising a poly(methyl methacrylate) backbone and comprising polyisobutylene grafts.

Non-limiting mention may be made, as grafted or sequential block copolymers comprising at least one block resulting from the polymerization of at least one ethylenic monomer and at least one block of a polyether, such as a polyoxy(C₂-C₁₈)alkylene, such as polyoxyethylene and/or polyoxypropylene, of polyoxyethylene/polybutadiene or polyoxyethylene/polyisobutylene bi- or trisequential copolymers.

Use may also be made, as the stabilizing agent, of compounds such as:
(d) alkyl dimethicones in which the alkyl group comprises 6 to 32 carbon atoms, such as lauryl methicone and stearyl methicone, for example Si tec LDM 3107 from ISP, cetyl dimethicone, such as Abil Wax 9801, behenoxy dimethicone, such as Abil 5440 from Goldschmidt.
(e) dimethiconol esters of formula: 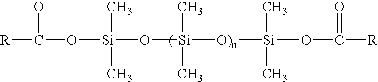
   in which R is an alkyl radical having 6 to 32 carbon atoms, such as dimethiconol behenate, and for example the products ULTRABEE from Noveon and PECOSIL DB from Phoenix Chemical.
(f) alkylaminoamines having for example 6 to 60 carbon atoms,such as 12 to 50, such as behenamidopropyldimethylamine and for example CATEMOL 220 from Phoenix Chemical, of formula: 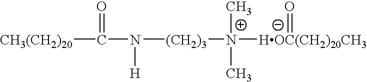
(g) copolymers comprising at least one polyorganosiloxane part and fluorinated groups, and for example fluorinated silicones or fluorosilicones which can be represented by the formula: 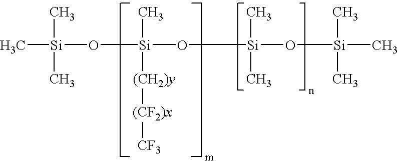
   in which x is an integer from 3 to 12, such as 5 to 10, for instance, x=8; y is an integer from 2 to 6, such as 2 or 3; and m and n are such that the molecular weight of the compound ranges from 5,000 to 15,000;
   and for example perfluorononyl dimethicones, such as those sold under the name PECOSIL FSH-150 and 300 or PECOSIL FSL-150 and 300 by Phoenix Chemical.

According to at least one embodiment, when the synthesis solvent is nonpolar, a polymer contributing the completest possible covering of the particles, several chains of stabilizing polymers then being adsorbed on one particle of polymer obtained by polymerization is chosen as the stabilizing agent.

In this embodiment, as stabilizing agent, either a grafted polymer or a sequential polymer, so as to have better interfacial activity may be chosen, for example. This is because the sequences or grafts which are insoluble in the synthesis solvent contribute more voluminous covering at the surface of the particles.

When the liquid synthesis solvent comprises at least one silicone oil, the stabilizing agent may be chosen from the group comprising grafted or sequential block copolymers comprising at least one block of polyorganosiloxane type and at least one block of a radical polymer or of a polyether or of a polyester, such as polyoxypropylenated and/or oxyethylenated blocks, for example.

For instance, the dispersion according to the present disclosure is such that:
the monomers with a Tg of less than or equal to −20° C. are chosen from, alone or as a mixture, ethyl, propyl, n-butyl, isobutyl, 2-ethylhexyl, octyl, isooctyl, isodecyl, decyl, lauryl, tridecyl, hydroxyethyl and hydroxypropyl acrylates; octyl, isooctyl, decyl, isodecyl, dodecyl, lauryl, tridecyl, myristyl, cetyl, palmityl, stearyl, behenyl and oleyl methacrylates, and comprise, for example, at least 2-ethylhexyl acrylate or methacrylate, and/or
the monomers with a Tg of less than or equal to −20° C. are present in an amount ranging from 50 to 100% by weight relative to the total weight of the starting monomers; and/or
the stabilizing agent is chosen from fluorinated silicones or fluorosilicones of formula: 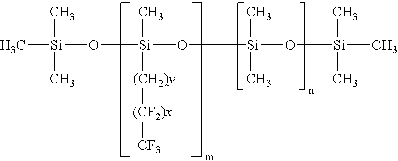
   in which x=8, y=2 or 3, and m and n are such that the molecular weight of the compound ranges from 5,000 to 15,000; and/or
the nonaqueous liquid compound is chosen from volatile silicone oils, for example cyclic or linear volatile silicone oils, such as cyclodimethylsiloxanes and linear dimethylsiloxanes, and/or esters of formula RCOOR′ in which R is the residue of a higher fatty acid comprising from 7 to 19 carbon atoms and R′ is a hydrocarbon chain comprising from 3 to 20 carbon atoms, such as palmitates, adipates, myristates and benzoates, for instance diisopropyl adipate and isopropyl myristate; and their mixtures.

It is possible to add at least one plasticizer to the dispersion of polymers, so as to lower the Tg of the polymers used. The at least one plasticizer can be chosen from the plasticizers conventionally used in the field of application, for example from the compounds capable of being solvents for the polymer. The at least one plasticizer can be incorporated during the synthesis or added once the synthesis has been carried out.

The dispersions obtained according to the present disclosure can then be used in a composition, for instance in a cosmetic or pharmaceutical composition, which furthermore comprises a cosmetically or pharmaceutically acceptable medium.

Depending on the application envisaged, use may be made of dispersions of film-forming or non-film-forming polymers in a nonaqueous medium comprising volatile or nonvolatile oils.

The composition can then comprise, depending on the application envisaged, the constituents standard to this type of composition.

Non-limiting mention may be made, among these constituents, of waxes, oils, gums and/or pasty fatty substances of vegetable, animal, mineral or synthetic, indeed even silicone, origin, and their mixtures.

Non-limiting mention may be made, among the waxes capable of being present in the composition according to the present disclosure, alone or as a mixture, of hydrocarbon waxes, such as beeswax; carnauba, candelilla, ouricury or Japan wax, cork fibre, or sugarcane waxes; paraffin, lignite waxes; microcrystalline waxes; lanolin wax; montan wax; ozokerites; polyethylene waxes; waxes obtained by Fischer-Tropsch synthesis; oils which are hydrogenated, fatty esters and glycerides which are solid at 25° C. Use may also be made of silicone waxes, including but not limited to alkyl derivatives, alkoxy derivatives and esters of polymethylsiloxane.

The composition according to the present disclosure can also comprise carbon, hydrocarbon, fluorinated and/or silicone oils of mineral, animal, vegetable or synthetic origin, alone or as a mixture, insofar as they form a homogeneous and stable mixture and insofar as they are compatible with the use envisaged. Non-limiting mention may be made, among the oils capable of being present in the composition according to the present disclosure, alone or as a mixture, of hydrocarbon oils, such as liquid paraffin or liquid petrolatum; perhydrosqualene; arara oil; sweet almond, calophyllum, palm, castor, avocado, jojoba, olive or cereal germ oil; esters of lanolic acid, of oleic acid, of lauric acid, of stearic acid; alcohols, such as oleyl alcohol, linoleyl or linolenyl alcohol, isostearyl alcohol or octyldodecanol. Non-limiting mention may also be made of silicone oils, such as PDMSs, which are optionally phenylated, such as phenyl trimethicones. Use may also be made of volatile oils, such as cyclotetradimethylsiloxane, cyclopentadimethylsiloxane, cyclohexadimethylsiloxane, methylhexyldimethylsiloxane, hexamethyldisiloxane or isoparaffins.

The composition according to the present disclosure can also comprise one or more coloring materials chosen from pulverulent compounds and/or fat-soluble or water-soluble dyes, for example in an amount ranging from 0 to 70% of the total weight of the composition, such as from 0.01 to 70% by weight, relative to the total weight of the composition.

The pulverulent compounds can be chosen from the pigments and/or pearlescent agents and/or fillers commonly used in cosmetic or pharmaceutical compositions. According to at least one embodiment, the pulverulent compounds may be present in an amount ranging from 0 to 50%, for instance from 0.1 to 50% of the total weight of the composition and further for example from 1 to 40% by weight, relative to the total weight of the composition.

The pigments can be white or colored, inorganic and/or organic and interferential or noninterferential. Non-limiting mention may be made, among inorganic pigments, of titanium dioxide, optionally treated at the surface, zirconium or cerium oxides, and also iron or chromium oxides, manganese violet, ultramarine blue, chromium hydrate and ferric blue. Non-limiting mention may be made, among organic pigments, of carbon black, pigments of D&C type and lakes based on cochineal carmine of barium, strontium, calcium, aluminium.

The pearlescent pigments can be chosen from white pearlescent pigments, such as mica covered with titanium oxide or with bismuth oxychloride, colored pearlescent pigments, such as titanium oxide-coated mica with iron oxides, titanium oxide-coated mica with, for instance, ferric blue or chromium oxide, titanium oxide-coated mica with an organic pigment of the abovementioned type, and also pearlescent pigments based on bismuth oxychloride.

The fillers can be inorganic or organic, and lamellar or spherical. Non-limiting mention may be made of talc, mica, silica, kaolin, Nylon and polyethylene, poly-β-alanine and polyethylene powders, Teflon, lauroyllysine, starch, boron nitride, tetrafluoroethylene polymer powders, hollow microspheres, such as EXPANCEL (Nobel Industrie) or POLYTRAP (Dow Corning), and silicone resin microbeads (TOSPEARLS from Toshiba, for example), precipitated calcium carbonate, magnesium carbonate, basic magnesium carbonate, hydroxyapatite, hollow silica microspheres (SILICA BEADS from Maprecos), glass or ceramic microcapsules, metal soaps derived from organic carboxylic acids having from 8 to 22 carbon atoms, such as from 12 to 18 carbon atoms, for example zinc stearate, magnesium stearate, lithium stearate, zinc laurate or magnesium myristate.

The fat-soluble dyes are, for example, Sudan red, D&C Red 17, D&C Green 6, β-carotene, soybean oil, Sudan brown, D&C Yellow 11, D&C Violet 2, D&C Orange 5 or quinoline yellow. They can be present in an amount ranging from 0 to 20%, for example 0.01 to 20% of the weight of the composition and further still, for example, from 0.1 to 6%. The water-soluble dyes are, for example, beetroot juice or methylene blue and can be present in an amount up to 6% of the total weight of the composition.

The composition can additionally comprise any additive conventionally used in the cosmetics field, such as antioxidants, fragrances, essential oils, preservatives, cosmetic active principles, moisturizing agents, vitamins, essential fatty acids, ceramides, sunscreens, surfactants, polymers, thickeners or gelling agents. Of course, a person skilled in the art will take care to choose this or these additives and/or their amount so that the beneficial properties of the composition according to the present disclosure are not, or not substantially, detrimentally affected by the envisaged addition.

The compositions according to the present disclosure can be provided in any form acceptable and conventional for a cosmetic or pharmaceutical composition.

The composition according to the present disclosure can be provided in the form of a suspension, a dispersion, such as of oil in water, by virtue of vesicles; an optionally thickened, indeed even gelled, oily solution; an oil-in-water, water-in-oil or multiple emulsion; a gel or a foam; an oily or emulsified gel; a dispersion of vesicles, for instance lipid vesicles; a two-phase or multiphase lotion; a spray; of a lotion, of a cream, of an ointment, of a soft paste, of a salve, of a cast or molded solid, for instance cast or molded as a stick or in a dish, or also of a compacted solid.

A person skilled in the art can choose the appropriate dosage form, and its method of preparation, on the basis of his general knowledge, taking into account, on the one hand, the nature of the constituents used, for instance their solubility in the vehicle, and, on the other hand, the application envisaged for the composition.

The cosmetic composition according to the present disclosure can be provided in the form of a product for caring for and/or making up the skin of the body or face, the lips and the hair, of an antisun or self-tanning product, indeed even of a hair product.

For instance, the cosmetic composition has beneficial application in the hair field, such as in the form retention of the hairstyle or the shaping of the hair. The hair compositions may be in the form of, for example, shampoos, gels, hair setting lotions, blow drying lotions, and fixing and styling compositions, such as lacquers, foams and sprays. The lotions can be packaged in various forms, such as in vaporizers, pump-action sprays and aerosol containers, in order to provide for application of the composition in the vaporized form or in the foam form.

The compositions as disclosed herein can be used for washing or treating keratinous substances, such as the hair, skin, eyelashes, eyebrows, nails, lips or scalp, for instance, the hair.

The compositions according to the present disclosure can be detergent compositions, such as shampoos, shower gels and foam baths.

The compositions of the present disclosure can also be provided in the form of rinse-out or leave-in conditioners, of perming, hair straightening, dyeing or bleaching compositions, or else in the form of rinse-out compositions, to be applied before or after a dyeing, bleaching, perming or hair straightening or else between the two stages of a perming or hair straightening.

The compositions of the present disclosure can also be provided in the form of washing compositions for the skin, for instance in the form of solutions or gels for the bath or shower or of make-up removers.

The compositions according to the present disclosure can also be provided in the form of aqueous or aqueous/alcoholic lotions for caring for the skin and/or hair.

Another aspect of the present disclosure is a method for the cosmetic treatment of keratinous substances, such as the skin of the body or face, the nails, the hair and/or the eyelashes, comprising the application, to the substances, of a cosmetic composition as defined above.

This method according to the present disclosure makes possible the form retention of the hairstyle or the treatment, care or washing of or removal of make-up from the skin, hair or any other keratinous substance.

The present disclosure is illustrated in more detail in the following examples, in which the Tg of the polymers is calculated according to the Fox law; when neutralized monomers are employed, the Tg is then “estimated” on the basis of the Tg of the homopolymer obtained from the nonneutralized monomer, given that it is known that the neutralization lowers the Tg when it is carried out with the neutralizing agents chosen.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in its respective testing measurement.

The examples that follow are intended to illustrate the present disclosure without, however, being limiting in nature.

### EXAMPLES

### Examples 1 to 46

Nonaqueous dispersions according to the present disclosure were prepared in the following way: the compounds mentioned below were introduced with stirring into a reactor. Heating was subsequently carried out at 90° C. for 6 hours.

| Compounds | | | | | | | | |
|---|---|---|---|---|---|---|---|---|
| (g) | Ex. 1 | Ex. 2 | Ex. 3 | Ex. 4 | Ex. 5 | Ex. 6 | Ex. 7 | Ex. 8 |
| 2-Ethylhexyl | 2.4 | 2.4 | 2.4 | 2.4 | 2.4 | 2.4 | 2.4 | 2.4 |
| acrylate | | | | | | | | |
| Tg = −50° C. | | | | | | | | |
| Acrylic acid | 0.05 | 0.05 | 0.05 | 0.05 | 0.05 | 0.05 | 0.05 | 0.05 |
| Tg = 105° C. | | | | | | | | |
| Silicone oil | 3 | 3 | 3 | 3 | 3 | 3 | 3 | 3 |
| (D5) | | | | | | | | |
| Stabilizing | 0.04 | 0.09 | | | | | | |
| agent 1 | | | | | | | | |
| Stabilizing | | | 0.04 | 0.09 | | | | |
| agent 2 | | | | | | | | |
| Stabilizing | | | | | 0.04 | 0.09 | | |
| agent 3 | | | | | | | | |
| Stabilizing | | | | | | | 0.04 | 0.09 |
| agent 4 | | | | | | | | |
| Initiator | 0.06 | 0.06 | 0.06 | 0.06 | 0.06 | 0.06 | 0.06 | 0.06 |
| Tg of the | −48° C. | −48° C. | −48° C. | −48° C. | −48° C. | −48° C. | −48° C. | −48° C. |
| polymer | | | | | | | | |
| (calculated) | | | | | | | | |

| Compounds | | | | | | |
|---|---|---|---|---|---|---|
| (g) | Ex. 9 | Ex. 10 | Ex. 11 | Ex. 12 | Ex. 13 | Ex. 14 |
| 2-Ethylhexyl | 2.4 | 2.4 | 2.4 | 2.4 | 2.4 | 2.4 |
| acrylate | | | | | | |
| Acrylic acid | 0.05 | 0.05 | 0.05 | 0.05 | 0.05 | 0.05 |
| Silicone oil | 3 | 3 | 3 | 3 | 3 | 3 |
| (D5) | | | | | | |
| Stabilizing | 0.04 | 0.09 | | | | |
| agent 5 | | | | | | |
| Stabilizing | | | 0.04 | 0.09 | | |
| agent 6 | | | | | | |
| Stabilizing | | | | | 0.04 | 0.09 |
| agent 7 | | | | | | |
| Initiator | 0.06 | 0.06 | 0.06 | 0.06 | 0.06 | 0.06 |
| Tg of the | −48° C. | −48° C. | −48° C. | −48° C. | −48° C. | −48° C. |
| polymer | | | | | | |
| (calculated) | | | | | | |

| Compounds | Ex. | Ex. | Ex. | Ex. | Ex. | Ex. | Ex. | Ex. |
|---|---|---|---|---|---|---|---|---|
| (g) | 15 | 16 | 17 | 18 | 19 | 20 | 21 | 22 |
| 2-Ethylhexyl | 2.4 | 2.4 | 2.4 | 2.4 | 2.4 | 2.4 | 2.4 | 2.4 |
| acrylate | | | | | | | | |
| AMP- | 0.05 | 0.05 | 0.05 | 0.05 | 0.05 | 0.05 | 0.05 | 0.05 |
| neutralized | | | | | | | | |
| acrylic acid | | | | | | | | |
| Silicone oil | 3 | 3 | 3 | 3 | 3 | 3 | 3 | 3 |
| (D5) | | | | | | | | |
| Stabilizing | 0.04 | 0.09 | | | | | | |
| agent 1 | | | | | | | | |
| Stabilizing | | | 0.04 | 0.09 | | | | |
| agent 2 | | | | | | | | |
| Stabilizing | | | | | 0.04 | 0.09 | | |
| agent 3 | | | | | | | | |
| Stabilizing | | | | | | | 0.04 | 0.09 |
| agent 4 | | | | | | | | |
| Initiator | 0.06 | 0.06 | 0.06 | 0.06 | 0.06 | 0.06 | 0.06 | 0.06 |
| Tg of the | less | less | less | less | less | less | less | less |
| polymer | than | than | than | than | than | than | than | than |
| (estimated) | −48° C. | −48° C. | −48° C. | −48° C. | −48° C. | −48° C. | −48° C. | −48° C. |

| Compounds | Ex. | Ex. | Ex. | Ex. | Ex. | Ex. | Ex. | Ex. |
|---|---|---|---|---|---|---|---|---|
| (g) | 23 | 24 | 25 | 26 | 27 | 28 | 29 | 30 |
| 2-Ethylhexyl | 2.4 | 2.4 | 2.4 | 2.4 | 2.4 | 2.4 | 2.4 | 2.4 |
| acrylate | | | | | | | | |
| AMP- | 0.05 | 0.05 | 0.05 | 0.05 | | | | |
| neutralized | | | | | | | | |
| acrylic acid | | | | | | | | |
| SPE monomer | | | | | 0.01 | 0.01 | 0.01 | 0.01 |
| Silicone oil | 3 | 3 | 3 | 3 | 3 | 3 | 3 | 3 |
| (D5) | | | | | | | | |
| Stabilizing | 0.04 | 0.09 | | | 0.04 | 0.09 | | |
| agent 5 | | | | | | | | |
| Stabilizing | | | 0.04 | 0.09 | | | 0.09 | 0.09 |
| agent 7 | | | | | | | | |
| Initiator | 0.06 | 0.06 | 0.06 | 0.06 | 0.06 | 0.06 | 0.06 | 0.06 |
| Tg of the | less | less | less | less | — | — | — | — |
| polymer | than | than | than | than | | | | |
| (estimated) | −48° C. | −48° C. | −48° C. | −48° C. | | | | |

| Compounds | Ex. | Ex. | Ex. | Ex. | Ex. | Ex. | Ex. | Ex. |
|---|---|---|---|---|---|---|---|---|
| (g) | 31 | 32 | 33 | 34 | 35 | 36 | 37 | 38 |
| 2-Ethylhexyl | 2.4 | 2.4 | 2.4 | 2.4 | 2.4 | 2.4 | 2.4 | 2.4 |
| acrylate | | | | | | | | |
| MADQUAT | 0.01 | 0.01 | 0.01 | 0.01 | 0.01 | 0.01 | 0.01 | 0.01 |
| N-octyl | | | | | | | | |
| Silicone oil | 3 | 3 | 3 | 3 | 3 | 3 | 3 | 3 |
| (D5) | | | | | | | | |
| Stabilizing | 0.04 | 0.09 | | | | | | |
| agent 1 | | | | | | | | |
| Stabilizing | | | 0.04 | 0.09 | | | | |
| agent 2 | | | | | | | | |
| Stabilizing | | | | | 0.04 | 0.09 | | |
| agent 5 | | | | | | | | |
| Stabilizing | | | | | | | 0.04 | 0.09 |
| agent 7 | | | | | | | | |
| Initiator | 0.06 | 0.06 | 0.06 | 0.06 | 0.06 | 0.06 | 0.06 | 0.06 |
| Tg of the | less | less | less | less | less | less | less | less |
| polymer | than | than | than | than | than | than | than | than |
| (estimated) | −48° C. | −48° C. | −48° C. | −48° C. | −48° C. | −48° C. | −48° C. | −48° C. |
| Compounds | Ex. | Ex. | Ex. | Ex. | Ex. | Ex. | Ex. | Ex. |
| (g) | 39 | 40 | 41 | 42 | 43 | 44 | 45 | 46 |
| 2-Ethylhexyl | 2.4 | 2.4 | 2.4 | 2.4 | 2.4 | 2.4 | 2.4 | 2.4 |
| acrylate | | | | | | | | |
| Neutralized | 0.01 | 0.01 | 0.01 | 0.01 | 0.01 | 0.01 | 0.01 | 0.01 |
| MADAME | | | | | | | | |
| Silicone oil | 3 | 3 | 3 | 3 | 3 | 3 | 3 | 3 |
| (D5) | | | | | | | | |
| Stabilizing | 0.04 | 0.09 | | | | | | |
| agent 1 | | | | | | | | |
| Stabilizing | | | 0.04 | 0.09 | | | | |
| agent 2 | | | | | | | | |
| Stabilizing | | | | | 0.04 | 0.09 | | |
| agent 5 | | | | | | | | |
| Stabilizing | | | | | | | 0.04 | 0.09 |
| agent 7 | | | | | | | | |
| Initiator | 0.06 | 0.06 | 0.06 | 0.06 | 0.06 | 0.06 | 0.06 | 0.06 |
| Tg of the | less | less | less | less | less | less | less | less |
| polymer | than | than | than | than | than | than | than | than |
| (estimated) | −49° C. | −49° C. | −49° C. | −49° C. | −49° C. | −49° C. | −49° C. | −49° C. |

Stabilizing agent 1: dimethicone copolyol (KF6017 from Shin-Etsu)
Stabilizing agent 2: dimethicone copolyol (DC5225C from Dow Corning)
Stabilizing agent 3: acrylate/dimethicone copolyol (KP 545 from Shin-Etsu)
Stabilizing agent 4: acrylates/stearyl acrylate/dimethicone acrylates copolymer (KP 561 from Shin-Etsu)
Stabilizing agent 5: polyoldimethicone block (Mw 8000) (GP675 from Genesee Polymers)
Stabilizing agent 6: mixture of cetyl dimethicone copolyol, of polyglyceryl isostearate (4 mol) and of hexyl laurate (ABIL WE-09 from Goldschmidt)
Stabilizing agent 7: perfluorononyl dimethicone (PECOSIL FSH-150 from Phoenix Chemical)
Initiator: tert-butyl peroxy(2-ethylhexanoate) (TRIGONOX 21S from Akzo)
SPE monomer: N,N-dimethyl-N-(2-methacryloyloxyethyl)-N-(3-sulphopropyl)ammonium betaine
MADQUAT N-octyl: N,N′,N″-octyldimethylammonioethyl methacrylate, with a Tg of less than 20° C.
Neutralized MADAME: dimethylaminoethyl methacrylate neutralized to 100% by ethylcaproic acid (MADAME has a Tg = 19° C.)

In all of the examples above, a dispersion of polymer particles in a volatile silicone oil was obtained in the end.

### Example 47

The constituents of the vessel heel were introduced into a 1 liter reactor equipped with a reflux condenser and a 250 ml dropping funnel. The temperature was set by external regulation at 90° C., with a rise in temperature of 2° C./minute, and mechanical stirring of 300 revolutions/min.

Once the required temperature was reached (90° C.), the constituents of the portion run in were added over 1 hour. The temperature was maintained at 90° C. for 5 hours.

| | Vessel heel | Portion run in | Total amount |
|---|---|---|---|
| | (g) | (g) | (g) |
| 2-Ethylhexyl acrylate | 18 | 80 | 98 |
| Stabilizing agent 7 | 2 | — | 2 |
| Silicone oil (D5) | 90 | 90 | 180 |
| Initiator | 2 | 0.6 | 2.6 |
| (Trigonox 21S) | | | |

A stable dispersion of poly(2-ethylhexyl acrylate) in a volatile silicone oil (D5) was obtained. The calculated Tg of the polymer was −50° C.

The measurement of the size of the particles, carried out by quasielastic light scattering with a Coulter N4 SD, provided the following results:
mean size of the particles: 150 nm

The number-average molecular weight, determined by GPC (elution solvent: THF), was 19 300 g/mol.
polydispersity: 5.

### Example 48

The constituents of the vessel heel were introduced into a 500 ml reactor equipped with a reflux condenser and a 250 ml dropping funnel. The temperature was set by external regulation at 90° C., with a rise in temperature of 2° C./minute, and mechanical stirring of 300 revolutions/min.

Once the required temperature was reached (90° C.), the constituents of the portion run in were added over 1 hour and 20 minutes. The temperature was maintained at 90° C. for 6 hours.

| | Vessel heel | Portion run in | Total amount |
|---|---|---|---|
| | (g) | (g) | (g) |
| 2-Ethylhexyl acrylate | — | 70 | 70 |
| MADAME | — | 30 | 30 |
| Ethylcaproic acid | — | 30 | 30 |
| Stabilizing agent 7 | 2 | 3 | 5 |
| Isopropyl myristate | 50 | 150 | 200 |
| Initiator | 0.5 | 1 | 1.5 |
| (Trigonox 21S) | | | |

A stable dispersion of particles of poly(2-ethylhexyl acrylate/MADAME neutralized to 100%) in isopropyl myristate was obtained. The estimated Tg of the polymer was less than −33° C.

### Example 49

The constituents of the vessel heel were introduced into a 500 ml reactor equipped with a reflux condenser and a 250 ml dropping funnel. The temperature was set by external regulation at 90° C., with a rise in temperature of 2° C./minute, and mechanical stirring of 300 revolutions/min.

Whitening was recorded approximately 30 minutes after the required temperature was reached (90° C.); the constituents of the portion run in were then added over 1 hour. The temperature was maintained at 90° C. for 3 hours.

| | Vessel heel | Portion run in | Total amount |
|---|---|---|---|
| | (g) | (g) | (g) |
| Methyl acrylate | 24 | 6 | 30 |
| (Tg = 10° C.) | | | |
| Butyl acrylate | 14 | 56 | 70 |
| (Tg = −54° C.) | | | |
| Stabilizing agent 2 | 5 | — | 5 |
| Silicone oil (D5) | 215 | — | 215 |
| Initiator | 1 | 1.6 | 2.6 |
| (Trigonox 21S) | | | |

A stable dispersion of particles of poly(methyl acrylate/butyl acrylate) in a silicone oil was obtained. The calculated Tg of the polymer was −38° C.

### Example 50

The constituents of the vessel heel were introduced into a 500 ml reactor equipped with a reflux condenser and a 250 ml dropping funnel. The temperature was set by external regulation at 90° C., with a rise in temperature of 2° C./minute, and mechanical stirring of 300 revolutions/min.

Whitening was recorded approximately 45 minutes after the required temperature was reached (90° C.); the constituents of the portion run in were then added over 50 minutes. The temperature was maintained at 90° C. for 3 hours.

| | Vessel heel | Portion run in | Total amount |
|---|---|---|---|
| | (g) | (g) | (g) |
| Methyl acrylate | 24 | 6 | 30 |
| (Tg = 10° C.) | | | |
| Butyl acrylate | 14 | 56 | 70 |
| (Tg = −54° C.) | | | |
| Stabilizing agent 7 | 15 | — | 15 |
| Silicone oil (D5) | 215 | — | 215 |
| Initiator | 1 | 1.6 | 2.6 |
| (Trigonox 21S) | | | |

A stable dispersion of particles of poly(methyl acrylate/butyl acrylate) in a silicone oil was obtained. The calculated Tg of the polymer was −38° C.

### Example 51

The constituents of the vessel heel were introduced into a 500 ml reactor equipped with a reflux condenser and a 250 ml dropping funnel. The temperature was set by external regulation at 90° C., with a rise in temperature of 2° C./minute, and mechanical stirring of 300 revolutions/min.

Whitening was recorded approximately 40 minutes after the required temperature was reached (90° C.); the constituents of the portion run in were then added over 1 hour and 10 minutes. The temperature was maintained at 90° C. for 3 hours.

| | Vessel heel | Portion run in | Total amount |
|---|---|---|---|
| | (g) | (g) | (g) |
| Methyl acrylate | 24 | 6 | 30 |
| (Tg = 10° C.) | | | |
| Butyl acrylate | 14 | 56 | 70 |
| (Tg = −54° C.) | | | |
| Stabilizing agent 7 | 15 | — | 15 |
| 1,3-Butanediol | — | 3 | 3 |
| dimethacrylate | | | |
| Silicone oil (D5) | 215 | — | 215 |
| Initiator | 1 | 1.6 | 2.6 |
| (Trigonox 21S) | | | |

A stable dispersion of particles of crosslinked poly(methyl acrylate/butyl acrylate) in a silicone oil was obtained. The calculated Tg of the polymer was −38° C.

### Example 52

The constituents of the vessel heel were introduced into a 500 ml reactor equipped with a reflux condenser and a 250 ml dropping funnel. The temperature was set by external regulation at 90° C., with a rise in temperature of 2° C./minute, and mechanical stirring of 300 revolutions/min.

Whitening was recorded approximately 30 minutes after the required temperature was reached (90° C.); the constituents of the portion run in were then added over 1 hour. The temperature was maintained at 90° C. for 5 hours.

| | Vessel | Portion run | |
|---|---|---|---|
| | heel (g) | in (g) | Total amount (g) |
| 2-Ethylhexyl acrylate | — | 98 | 98 |
| Dodecafluoroheptyl | — | 2 | 2 |
| methacrylate (Tg ≦ 50° C.) | | | |
| Stabilizing agent 7 | 5 | — | 5 |
| Silicone oil (D5) | 200 | — | 200 |
| Initiator | 1 | 1.6 | 2.6 |
| (Trigonox 21S) | | | |

A stable dispersion of particles of poly(2-ethylhexyl acrylate/dodecafluoro-heptyl methacrylate) in a silicone oil was obtained. The estimated Tg of the polymer was less than −48° C.

### Example 53

A composition comprising the following constituents was compared (the percentages were expressed as percentage by weight of active material):

| | Composition | | | Comparative |
|---|---|---|---|---|
| | according to | Control | Comparative | composition |
| Constituent | the invention | composition | composition I | II |
| Dispersion of | 1.0 | — | — | — |
| Example 47 | | | | |
| Nonaqueous | — | — | 1.0 | — |
| dispersion of | | | | |
| polymer A | | | | |
| Nonaqueous | — | — | — | 1.0 |
| solution of | | | | |
| polymer B | | | | |
| Cyclopentasiloxane | 0.5 | 0.5 | 0.5 | 0.5 |
| dimethicone | | | | |
| copolyol | | | | |
| Cyclopentasiloxane | 10.0 | 10.0 | 10.0 | 10.0 |
| D5 | | | | |
| Trimethylbehenylammonium | 1.2 | 1.2 | 1.2 | 1.2 |
| chloride | | | | |
| Propylene glycol | 2.5 | 2.5 | 2.5 | 2.5 |
| Preservative | q.s. | q.s. | q.s. | q.s. |
| Fragrance | q.s. | q.s. | q.s. | q.s. |
| Citric acid/sodium | q.s. for | q.s. for | q.s. for | q.s. for |
| hydroxide | pH 6.5 | pH 6.5 | pH 6.5 | pH 6.5 |
| Water | q.s. 100 | q.s. 100 | q.s. 100 | q.s. 100 |

Dispersion A: dispersion of copolymer of methyl acrylate (90% by weight) and acrylic acid (10% by weight) at 25% in cyclopentadimethylsiloxane (D5). The dispersion was stabilized by 2% of polymethylcetyl dimethyl methylsiloxane which was oxyethylenated (ABIL EM 90 from Goldschmidt). Calculated Tg of the polymer: 17° C.

Solution B: 50% solution of poly(2-ethylhexyl acrylate) in cyclopentadimethylsiloxane to which was added, after polymerization, 2% of polydimethylsiloxane comprising perfluorononyl grafts (PECOSIL FSH-150). Calculated Tg of the polymer: −50° C.

2 grams of each composition were applied to locks of natural Caucasian hair with a length of 20 cm weighing 2.7 grams. The locks were massaged, left to stand for 5 minutes and then rinsed out. The wet locks were wound around curlers with a diameter of 2 cm and were subsequently dried under a hairdryer at 70° C. for 30 minutes.

After drying, the tonicity of the curl, the feel of the hair and the disentangling of the locks using a comb were evaluated.

It is noted that the 3 formulations contributed a similar level of tonicity to the curl which was much better than that obtained with the control lock. The cosmetic qualities of feel and the disentangling differed greatly from one lock to another.

The lock treated by the composition according to the present disclosure had a similar feel to that of the lock treated by the control. The lock was soft, smooth and nongreasy and disentangling was easy.

In comparison with the lock treated by the composition according to the present disclosure, the lock treated by the composition outside the comparative composition I had a rough feel and disentangling was difficult.

In comparison with the lock treated by the composition according to the present disclosure, the lock treated by the composition outside the comparative composition II had a greasy and tacky feel.

In conclusion, only the composition according to the present disclosure made it possible to obtain good cosmetic properties of feel and disentangling while retaining a good level of styling.

What is claimed is:

1. A dispersion of particles of at least one ethylenic polymer, wherein the dispersion is stabilized at its surface by a stabilizing agent in a nonaqueous medium comprising at least one nonaqueous compound, liquid at 25° C., having an overall solubility parameter according to the Hansen solubility space of less than or equal to 20 (MPa)^{1/2}, and mixtures of such compounds;
wherein the at least one ethylenic polymer exhibits a glass transition temperature (Tg) that ranges from −99° C. to −20° C.

2. The dispersion according to claim 1, wherein the at least one ethylenic polymer exhibits a glass transition temperature (Tg) that ranges from −99° C. to −25° C.

3. The dispersion according to claim 2, wherein the at least one ethylenic polymer exhibits a glass transition temperature (Tg) that ranges from −70° C. to −40° C.

4. The dispersion according to claim 1, wherein the dispersion is provided in the form of polymer nanoparticles in dispersion in the nonaqueous medium, wherein the said nanoparticles range in size from 5 to 600 nm.

5. The dispersion according to claim 4, wherein the dispersion is provided in the form of polymer nanoparticles in dispersion in the nonaqueous medium, wherein the said nanoparticles range in size from 15 to 450 nm.

6. The dispersion according to claim 1, wherein the at least one ethylenic polymer results from the polymerization of at least one monomer with a Tg of less than or equal to −20° C., wherein the at least one ethylenic polymer is present in an amount of 100% by weight relative to the total weight of the monomers.

7. The dispersion according to claim 1, wherein the at least one ethylenic polymer results from the polymerization of at least one monomer with a Tg of less than or equal to −20° C. and of at least one additional monomer with a Tg of greater than −20° C.

8. The dispersion according to claim 7, wherein the at least one additional monomer, or the mixture of such monomers, is present in an amount ranging from 0.01 to 50% by weight, with respect to the total weight of the monomers; and the at least one monomer with a Tg of less than or equal to −20° C., or the mixture of such monomers, is present in an amount ranging from 50 to 99.99% by weight, with respect to the total weight of monomers.

9. The dispersion according to claim 8, wherein the at least one additional monomer, or the mixture of such monomers, is present in an amount ranging from 5 to 15% by weight, with respect to the total weight of the monomers; and the at least one monomer with a Tg of less than or equal to −20° C., or the mixture of such monomers, is present in an amount ranging from 85 to 95% by weight, with respect to the total weight of monomers.

10. The dispersion according to claim 1, wherein the at least one ethylenic polymer comprises at least one hydrophobic monomer present in the amount of 40% to 100% by weight, with respect to the total weight of monomers, alone or as a mixture.

11. The dispersion according to claim 10, wherein the at least one ethylenic polymer comprises at least one hydrophobic monomer present in the amount of 60 to 95% by weight, with respect to the total weight of monomers, alone or as a mixture.

12. The dispersion according to claim 10, wherein the at least one hydrophobic monomer has a Tg of less than −20° C.

13. The dispersion according to claim 6, wherein the at least one monomer with a Tg of less than or equal to −20° C. is chosen from, alone or as a mixture:
(i) esters of acrylic acid of formula CH₂═CHCOOR1 wherein R1 is (a) a saturated or unsaturated and linear or branched carbon chain, optionally comprising, inserted, at least one heteroatom chosen from O, N, S; and/or optionally substituted by at least one substituent chosen from —OH and halogen atoms chosen from Cl, Br, I and F, with the exception of the tert-butyl chain; or else R1 is (b) a polyoxyethylene group comprising from 5 to 30 ethylene oxide units; or also R1 is (c) a —R—(OC₂H₄)ₙ—H group with R═C₁-C₁₂ alkyl and n is an integer from 5 to 30 inclusive;
(ii) esters of methacrylic acid of formula CH₂═C(CH₃)COOR2 wherein R2 is (a) a saturated or unsaturated and linear or branched carbon chain, optionally comprising, inserted, at least one heteroatom chosen from O, N, S; and/or optionally substituted by at least one substituent chosen from —OH and halogen atoms chosen from Cl, Br, I and F; or else R2 is (b) a polyoxyethylene group comprising from 5 to 30 ethylene oxide units; or also R2 is (c) a —R—(OC₂H₄)ₙ—H group with R═C₁-C₃₀ alkyl and n is an integer from 5 to 30 inclusive;
(iii) vinyl esters of formula CH₂═CH—OCO—R3 wherein R3 is a saturated or unsaturated and linear or branched carbon chain;
(iv) vinyl ethers of formula CH₂═CHOR4 wherein R4 is a saturated or unsaturated and linear or branched carbon chain;
(v) N-alkyl(meth)acrylamides of formula CH₂═CHCONR5R′5 or CH₂═C(CH₃)CONR5R′5 wherein R5 and R′5 are, independently of one another, a hydrogen atom or a saturated or unsaturated and linear, cyclic or branched carbon chain, optionally aromatic chain (aryl, aralkyl or alkylaryl), optionally comprising, inserted, at least one heteroatom chosen from O, N, S; and/or optionally substituted by at least one substituent chosen from —OH and halogen atoms chosen from Cl, Br, I and F; given that at least one of the R5 and R′5 radicals is other than hydrogen.

14. The dispersion according to claim 13, wherein the at least one monomer with a Tg of less than or equal to −20° C. is chosen from, alone or as a mixture:
ethyl, propyl, n-butyl, isobutyl, 2-ethylhexyl, octyl, isooctyl, isodecyl, decyl, lauryl, tridecyl, hydroxyethyl and hydroxypropyl acrylates;
octyl, isooctyl, decyl, isodecyl, dodecyl, lauryl, tridecyl, myristyl, cetyl, palmityl, stearyl, behenyl and oleyl methacrylates;
vinyl butyrate, vinyl butanoate, vinyl ethylhexanoate, vinyl neononanoate and vinyl neododecanoate;
vinyl ether, methyl vinyl ether, ethyl vinyl ether, ethylhexyl vinyl ether and butyl vinyl ether;
N-octylacrylamide and N-octadecylacrylamide.

15. The dispersion according to claim 13, wherein the at least one monomer with a Tg of less than or equal to −20° C. is chosen from n-butyl, isobutyl, 2-ethylhexyl, octyl, isooctyl, isodecyl, decyl and lauryl acrylates; and also octyl, isooctyl, decyl, isodecyl, dodecyl, lauryl, tridecyl, cetyl, palmityl, stearyl and oleyl methacrylates; and their mixtures.

16. The dispersion according to claim 7, wherein the at least one additional monomer is chosen from, alone or as a mixture:
(i) vinyl compounds of formula CH₂═CHR6 in which R6 is
a hydroxyl group;
a linear or branched alkyl group comprising 1 to 25 carbon atoms, in which is optionally inserted at least one heteroatom chosen from O, N, S and P; it being possible for said alkyl group in addition to be optionally substituted by at least one substituent chosen from —OH and halogen atoms chosen from Cl, Br, I and F;
a C₃ to C₈ cycloalkyl group,
a C₆ to C₂₀ aryl group,
a C₇ to C₃₀ aralkyl group, wherein the alkyl portion is a C₁ to C₄ alkyl group,
a 4- to 12-membered heterocyclic group comprising at least one heteroatom chosen from O, N and S,
a heterocycloalkyl group (alkyl of 1 to 4 carbons),
it being possible for the said cycloalkyl, aryl, aralkyl, heterocyclic or heterocycloalkyl groups to be optionally substituted by at least one substituent chosen from the hydroxyl group, halogen atoms and linear or branched C₁-C₄ alkyl groups in which is optionally inserted at least one heteroatom chosen from O, N, S and P, it being possible for said alkyl groups in addition to be optionally substituted by at least one substituent chosen from —OH and halogen atoms chosen from Cl, Br, I and F;
(ii) acrylates of formula CH₂═CHCOOR7 in which R7 is a tert-butyl group; a C₃ to C₈ cycloalkyl group; a C₆ to C₂₀ aryl group; a C₇ to C₃₀ aralkyl group (C₁ to C₄ alkyl group); a 4- to 12-membered heterocyclic group comprising at least one heteroatom chosen from O, N and S; a heterocycloalkyl group (C₁ to C₄ alkyl); it being possible for said cycloalkyl, aryl, aralkyl, heterocyclic or heterocycloalkyl groups to be optionally substituted by at least one substituent chosen from the hydroxyl group, halogen atoms and linear or branched C₁-C₄ alkyl groups in which is optionally inserted at least one heteroatom chosen from O, N, S and P, it being possible for said alkyl groups in addition to be optionally substituted by at least one substituent chosen from the hydroxyl group and halogen atoms chosen from Cl, Br, I and F;
(iii) methacrylates of formula CH₂═C(CH₃)COOR8 in which R8 is:
a linear or branched carbon group, it being possible for said alkyl group in addition to be optionally substituted by at least one substituent chosen from OH and halogen atoms chosen from Cl, Br, I and F;
a C₃ to C₈ cycloalkyl group;
a C₆ to C₂₀ aryl group;
a C₇ to C₃₀ aralkyl group wherein the alkyl portion is a C₁ to C₄ alkyl group;
a 4- to 12-membered heterocyclic group comprising at least one heteroatom chosen from O, N and S;
a heterocycloalkyl group (C₁-C₄ alkyl);
it being possible for said cycloalkyl, aryl, aralkyl, heterocyclic or heterocycloalkyl groups to be optionally substituted by at least one substituent chosen from OH, halogen atoms and linear or branched C₁-C₄ alkyl groups in which is optionally inserted at least one heteroatom chosen from O, N, S and P, it being possible for said alkyl groups in addition to be optionally substituted by at least one substituent chosen from hydroxyl groups and halogen atoms chosen from Cl, Br, I and F;
(iv) (meth)acrylamides of formula CH₂═CHCONR9R′9 or CH₂═C(CH₃)CONR9R′9 in which R9 and R′9, which are identical or different, are each chosen from a hydrogen atom and a linear or branched C₁-C₅ alkyl group.

17. The dispersion according to claim 16, wherein the at least one additional monomer is chosen from vinylcyclohexane, styrene and vinyl acetate; tert-butyl, t-butylcyclohexyl, t-butylbenzyl, furfuryl and isobornyl acrylates; methyl, ethyl, propyl, n-butyl, isobutyl, t-butylcyclohexyl, t-butylbenzyl, methoxyethyl, methoxypropyl and isobornyl methacrylates; N-butyl(meth)acrylamide, N-isopropyl(meth)acrylamide, N,N-dimethyl(meth)acrylamide and N,N-dibutyl(meth)acrylamide, and their mixtures.

18. The dispersion according to claim 1, wherein the at least one ethylenic polymer has a number-average molecular weight (Mn) that ranges from 2,000 to 1,000,000.

19. The dispersion according to claim 18, in which the at least one ethylenic polymer has a number-average molecular weight (Mn) that ranges from 4,000 and 500,000.

20. The dispersion according to claim 1, wherein said nonaqueous liquid compound having an overall solubility parameter according to the Hansen solubility space of less than or equal to 20 (MPa)^{1/2}, is chosen from, alone or as a mixture, liquid fatty substances.

21. The dispersion according to claim 20, wherein the liquid fatty substances are branched, natural or synthetic, carbon, hydrocarbon, fluorinated and/or silicone oils.

22. The dispersion according to claim 1, wherein said nonaqueous liquid compound is chosen from:
vegetable oils formed by esters of fatty acids and of polyols;
hydrocarbons;
silicone oils, which are optionally substituted by aliphatic and/or aromatic groups which are optionally fluorinated or by functional groups; and volatile silicone oils, cyclophenylmethylsiloxanes and linear dimethylsiloxanes;
solvents, alone or as a mixture, chosen from linear, branched or cyclic esters having 6 to 30 carbon atoms; ethers having 6 to 30 carbon atoms and ketones having 6 to 30 carbon atoms;
aliphatic fatty monoalcohols having at least 6 carbon atoms, wherein the hydrocarbon chain does not comprise a substituent group.

23. The dispersion according to claim 1, wherein said nonaqueous liquid compound is chosen from volatile silicone oils, and/or esters of formula RCOOR′ in which R is the residue of a higher fatty acid comprising from 7 to 19 carbon atoms and R′ is a hydrocarbon chain comprising from 3 to 20 carbon atoms.

24. The dispersion according to claim 1, wherein the stabilizing agent is chosen from sequential polymers, grafted polymers and/or random polymers, alone or as a blend.

25. The dispersion according to claim 1, wherein the stabilizing agent is present in an amount ranging from 0.1 to 30% by weight, with respect to the weight of the starting mixture of monomers.

26. The dispersion according to claim 24, wherein the stabilizing agent is present in an amount ranging from 3 to 10% by weight, with respect to the weight of the starting mixture of monomers.

27. The dispersion according to claim 1, wherein the stabilizing agent is chosen from:
silicone polymers grafted with a hydrocarbon chain and hydrocarbon polymers grafted with a silicone chain;
grafted copolymers having an insoluble backbone of polyacrylic type with soluble grafts of poly(12-hydroxystearic acid) type;
grafted or sequential block copolymers comprising at least one block of polyorganosiloxane type and at least one block of a polymer (i) resulting from radical polymerization or (ii) resulting from polycondensation of polyether, polyester or polyamide type, or their blend, it being possible for said copolymer to comprise fluorinated entities;
grafted or sequential block copolymers of C₁-C₄ alkyl (meth)acrylates and of C₈-C₃₀ alkyl(meth)acrylates;
grafted or sequential block copolymers comprising at least one block resulting from the polymerization of ethylenic monomer, comprising at least one optionally conjugated ethylenic bond of dienes; and at least one block of polymer resulting from radical polymerization other than diene;
alkyl dimethicones in which the alkyl group comprises 6 to 32 carbon atoms;
dimethiconol esters of formula: 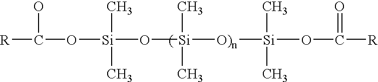
in which R is an alkyl radical having 6 to 32 carbon atoms;
alkylamidoamines;
copolymers comprising at least one polyorganosiloxane part and fluorinated groups which can be represented by the formula: 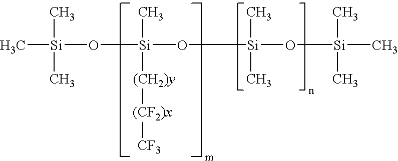
in which x is an integer ranging from 3 to 12; y is an integer ranging from 2 to 6; and m and n are such that the molecular weight of the compound ranges from 5,000 to 15,000;
grafted or sequential block copolymers comprising at least one block of polyorganosiloxane type and at least one polyether block;
dimethicone copolyols or also (C₂-C₁₈)alkyl methicone copolyols which are optionally crosslinked; lauryl dimethicone copolyol crosspolymer, cetyl dimethicone copolyol and dimethicone copolyol PPG-3 oleyl ether;
diblock or triblock copolymers chosen from polystyrene/polyisoprene, polystyrene/polybutadiene, polystyrene/copoly(ethylene-propylene), and polystyrene/copoly(ethylene-butylene);
poly(methyl methacrylate)/polyisobutylene bi- or trisequential copolymers or grafted copolymers comprising a poly(methyl methacrylate) backbone and comprising polyisobutylene grafts;
grafted or sequential block copolymers comprising at least one block resulting from the polymerization of at least one ethylenic monomer and at least one block of a polyether.

28. The dispersion according to claim 1, wherein the stabilizing agent is chosen from fluorinated silicones or fluorosilicones of formula: 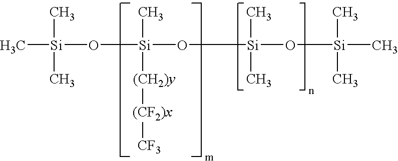
in which x=8, y=2 or 3, and m and n are such that the molecular weight of the compound ranges from 5,000 to 15,000.

29. A cosmetic or pharmaceutical composition comprising, in a cosmetically or pharmaceutically acceptable medium:
at least one dispersion of particles of at least one ethylenic polymer,
wherein the dispersion is stabilized at its surface by a stabilizing agent in a nonaqueous medium comprising at least one nonaqueous compound, liquid at 25° C., having an overall solubility parameter according to the Hansen solubility space of less than or equal to 20 (MPa)^{1/2}, and mixtures of such compounds;
wherein the at least one ethylenic polymer exhibits a glass transition temperature (Tg) that ranges from −99° C. to −20° C.

30. The composition according to claim 29, furthermore comprising at least one constituent chosen from waxes, oils, gums and/or pasty fatty substances of vegetable, animal, mineral or synthetic, or silicone, origin, and their mixtures; at least one coloring material chosen from pulverulent compounds and/or fat-soluble or water-soluble dyes; antioxidants, fragrances, essential oils, preservatives, cosmetic active principles, moisturizing agents, vitamins, essential fatty acids, ceramides, sunscreens, surfactants, polymers, thickeners and gelling agents.

31. The composition according to claim 29, which is provided in the form of a suspension, a dispersion; an optionally thickened, oily solution; an oil-in-water, water-in-oil or multiple emulsion; a gel or a foam; an oily or emulsified gel; a dispersion of vesicles, a two-phase or multiphase lotion; a spray; of a lotion, of a cream, of an ointment, of a soft paste, of a salve, of a cast or molded solid, or also of a compacted solid.

32. The composition according to claims 29, which is provided in the form of a product for caring for and/or making up the skin of the body or face, the lips and the hair, of an antisun or self-tanning product; of a hair product for the treatment, washing, form retention or shaping of the hair.

33. The composition according to claim 29, which is provided in the form of a shampoo, gel, hair setting lotion, blow drying lotion, or fixing and styling composition of a rinse-out or leave-in conditioner, of a perming, hair-straightening, dyeing or bleaching composition, or of a rinse-out composition, to be applied before or after dyeing, bleaching, perming or hair straightening or else between the two stages of a perming or hair straightening; of washing compositions for the skin; of aqueous or aqueous/alcoholic lotions for caring for the skin and/or hair.

34. A method for the cosmetic treatment of keratinous substances, such as of the skin of the body or face, the nails, the hair and/or the eyelashes, comprising:
applying to the keratinous substances a cosmetic or pharmaceutical composition;
wherein the cosmetic or pharmaceutical composition comprises, in a cosmetically or pharmaceutically acceptable medium:
at least one dispersion of particles of at least one ethylenic polymer,
wherein the dispersion is stabilized at its surface by a stabilizing agent in a nonaqueous medium, comprising at least one nonaqueous compound, liquid at 25° C., having an overall solubility parameter according to the Hansen solubility space of less than or equal to 20 (MPa)^{1/2}, and mixtures of such compounds;
wherein the at least one ethylenic polymer exhibits a glass transition temperature (Tg) that ranges from −99° C. to −20° C.
